(19) United States
(12) Patent Application Publication
(10) Pub. No.: US 2015/0031018 A1
(43) Pub. Date: Jan. 29, 2015

(54) Methods For Diagnosing Feline Coronavirus Infections

(71) Applicants: Gary Whittaker, Ithaca, NY (US); Andrew Regan, Athens, GA (US)

(72) Inventors: Gary Whittaker, Ithaca, NY (US); Andrew Regan, Athens, GA (US)

(21) Appl. No.: 14/496,819

(22) Filed: Sep. 25, 2014

Related U.S. Application Data
(62) Division of application No. 13/582,920, filed on Nov. 12, 2012.
(60) Provisional application No. 61/311,639, filed on Mar. 8, 2010.
(60) Provisional application No. 61/442,014, filed on Feb. 11, 2011.

(51) Int. Cl. C12Q 1/70; G01N 33/569

(52) U.S. Cl. CPC C12Q 1/701; G01N 33/56983; C12Q 2600/158; G01N 2333/165
USPC 435/5

(57) ABSTRACT

Provided is a method for determining whether a feline is infected with pathogenic Feline Infectious Peritonitis Virus (FIPV) or Feline Enteric Infection Virus (FECV). The method involves determining the presence or absence of intact or mutated S1/S2 and S2′ cleavage sites in the spike protein of serotype 1 feline coronaviruses (FCoV1). The presence of both intact cleavage sites is indicative of FECV. The presence of a mutation in one or both cleavage sites is indicative of FIPV. The absence of both sites is indicative of an absence of FCoV1 infection. Compositions for use in determining infection and kits are also provided.

### CROSS-REFERENCE TO RELATED APPLICATIONS

This application claims priority to U.S. Provisional Patent Application No. 61/311,639, filed on Mar. 8, 2010, and U.S. Provisional Patent Application No. 61/442,014, filed on Feb. 11, 2011, and to PCT/US11/027553, filed Mar. 8, 2014, and is a divisional of U.S. application Ser. No. 13/582,920, filed Nov. 12, 2012, the disclosures of each of which are incorporated herein by reference.

### FIELD OF THE INVENTION

The present invention relates generally to diagnosis of infectious viral disease in felines, and more particularly to diagnosing feline infectious peritonitis.

### BACKGROUND OF THE INVENTION

Feline infectious peritonitis (FIP) is a fatal, progressive, and immune-augmented disease of cats, which is of particular importance in shelter situations. It is caused by a group I feline coronavirus (FCoV1), which is also more recently referred to as Alphacoronavirus, subgroup Gesalavirus.

Although FCoV1 infection is wide-spread in domestic, feral and non-domestic cat populations world-wide (seroprevalence from 20-100%), less than 10% of FCoV1 seropositive cats develop FIP. FIP tends to occur most frequently in cats less than two years of age or, less commonly, in geriatric cats. The clinical manifestation of FCoV1 infection can present either as the pathogenic disease manifestation FIP (Feline Infectious Peritonitis Virus (FIPV)-cases) or the more common, benign or mild enteric infection (Feline Enteric Infection Virus (FECV)-asymptomatic). Specific genetic determinants of these clinical outcomes have yet to be discovered. There is no effective treatment or vaccine for FIP, nor a diagnostic protocol that can discriminate the avirulent from the pathogenic forms of FCoV1, known as FECV and FIPV, respectively. Cats infected with FECV that show no evidence of disease are thought to represent carriers of FCoV1 and may pose an FIP risk to other cats.

Based on serological differences, FCoV1 strains have been separated into a common type 1 form (80-90% prevalent in infected cats) and a less common type 2 form. FCoV1 types 1 and 2 appear to utilize distinct cell entry receptors and display different growth characteristics in vitro, due to the presence of different spike (S) genes. Both virulent and avirulent FCoV1 strains are found within types 1 and 2.

Viral genetic determinants that are specifically associated with FIPV pathogenesis have yet to be discovered. An “in vivo mutation transition hypothesis” also called the “internal mutation hypothesis” postulated that de novo mutation occurs in vivo giving rise to virulent forms of the virus which are able to spread systemically and lead to FIP pathogenesis. Haan et al. (J. Virol. (2008), Vol. 82, p. 6078-6083) described a furin enzyme recognition motif in FCoV spike protein that could be cleaved in certain cases, but conceded that there were no indications as to how furin cleavability of spike protein could somehow contribute to the virulent phenotype of FCoVs, and that the lack of cleavage of the FCoV UCD1 spike protein is not necessarily associated with high virulence. Hahn et al. did not disclose other FCoV1 spike mutations as being related to pathogenicity, and the complete mutation profile responsible for pathogenesis remains unidentified. Thus, there is an ongoing and unmet need for diagnostic tools that can be used for detecting FCoV1 infection and for discriminating FECV from FIPV. The present invention meets these and other related needs.

### SUMMARY OF THE INVENTION

The present invention provides methods and compositions for determining whether a feline is infected with FIPV or FECV. The method involves determining the presence or absence of intact or mutated S1/S2 and S2′ cleavage sites in the spike protein of serotype 1 feline coronaviruses (FCoV1). The invention also includes determining that a feline is not infected with FIPV or FECV.

In one aspect, the invention comprises determining from a biological sample obtained and/or derived from a feline the presence or absence of a mutation in the S1/S2 site in FCoV1 spike protein. The S1/S2 site is Arg Arg Ser Arg Arg Ser (SEQ ID NO:5). The method also involves determining the presence or absence of a mutation of the S2′ dibasic site in FCoV1 spike protein. The S2′ site is Xaa Xaa Ser Xaa Xaa Glu Asp Leu Leu Phe, where the dibasic site is comprised of the first two Xaa's (positions 1 and 2 in SEQ ID NO:8). Thus, positions 1 and 2 in SEQ ID NO:8 can be any two basic amino acids in sequence when no mutation is present. Accordingly, when reference to a mutation to the S2′ site is made herein, the mutation is a change in one of or both of the amino acids in positions 1 and 2 of SEQ ID NO:8 such that a dibasic site is no longer present. The second two Xaa's in SEQ ID NO:8 (positions 4 and 5) can be any amino acid. In one embodiment, they are Ala and Val, respectively. In another embodiment, they are Thr and Val, respectively.

If a mutation is present in the S1/S2 site, the S2′ site, or a combination thereof, the feline is identified as being infected with FIPV. For the S2′ site, informative mutations are those that alter the Xaa Xaa in positions 1 and 2 of SEQ ID NO:8, where the mutation(s) results in Xaa Xaa no longer being a dibasic amino acid couplet. Changes in positions 4 and 5 of SEQ ID NO:8 are not considered mutations to the S2′ site for purposes of determining infection with FIPV or FECV.

If non-mutated S1/S2 and S2′ sites are present, the feline is identified as infected with FECV. A lack of mutated and non-mutated S1/S2 and S2′ sites is indicative that the feline is not infected with FECV or FIPV.

The invention includes determining the presence and/or the absence of mutations of the S1/S2 and S2′ sites by analysis of nucleic acids encoding FCoV1 spike protein, as well as by analysis of FCoV1 spike protein itself. Analysis of nucleic acids includes direct analysis of viral RNA and analysis of polynucleotides amplified from viral RNA. Any nucleic acid analysis technique that can be used to determine the sequence of a nucleic acid can be used to perform the method of the invention. Analysis of the FCoV1 spike protein can be performed using, for example, immunodetection techniques involving antibodies that are developed for specifically discriminating mutated and non-mutated forms of FCoV1 spike protein, and/or cleaved or non-cleaved forms of the spike protein.

In another aspect, the present invention provides for a composition for determining whether a feline is infected with pathogenic FIPV or FECV comprising: a) a polynucleotide sequence for determining the presence or absence of a mutation in SEQ ID NO:5 (Arg Arg Ser Arg Arg Ser) in a FCoV1 spike protein; and b) a polynucleotide sequence for determining the presence or absence of a mutation of the dibasic couplet in the first two positions of SEQ ID NO:8 (Xaa Xaa Ser Xaa Xaa Glu Asp Leu Leu Phe) in the type 1 FCoV1 spike protein, wherein Xaa in the first two amino acids each comprise a basic amino acid and wherein the mutation is a change to a non-basic amino acid in the first, second or both Xaa in the first two positions of SEQ ID NO:8.

In still another aspect, the present invention provides for a kit for determining whether a feline is infected with pathogenic FIPV or FECV, in a single container or separate containers, comprising:

a) a polynucleotide sequence for determining the presence or absence of a mutation in SEQ ID NO:5 (Arg Arg Ser Arg Arg Ser) in FCoV1 spike protein; and

b) a polynucleotide sequence for determining the presence or absence of a mutation of SEQ ID NO:8 (Xaa Xaa Ser Xaa Xaa Glu Asp Leu Leu Phe) in FCoV1 spike protein, wherein Xaa in the first two positions of SEQ ID NO:8 is a basic amino acid and wherein the mutation is a change to a non-basic amino acid in the first, second or both of the Xaa's in the first two positions of SEQ ID NO:8.

In yet another aspect, the present invention provides for a composition for determining whether a feline is infected with FIPV or FECV comprising: a) a specific binder that can distinguish a FCoV1 polypeptide comprising an amino acid sequence set forth in SEQ ID NO:5 (Arg Arg Ser Arg Arg Ser) from a FCoV1 polypeptide comprising a mutation in the amino acid sequence set forth in SEQ ID NO:5; and b) a specific binder that can distinguish a FCoV1 polypeptide comprising an amino acid sequence set forth in SEQ ID NO:8 from a FCoV1 polypeptide comprising a mutation in the amino acid residue(s) Xaa and/or Xaa set forth in the first two positions of SEQ ID NO:8.

In yet another aspect, the present invention provides for a kit for determining whether a feline is infected with FIPV or FECV, in a single container or separate containers, comprising: a) a specific binder that can distinguish a FCoV1 polypeptide comprising an amino acid sequence set forth in SEQ ID NO:5 (Arg Arg Ser Arg Arg Ser) from a FCoV1 polypeptide comprising a mutation in the amino acid sequence set forth in SEQ ID NO:5; and b) a specific binder that can distinguish a FCoV1 polypeptide comprising an amino acid sequence set forth in SEQ ID NO:8 from a FCoV1 polypeptide comprising a mutation in the amino acid residue(s) Xaa and/or Xaa set forth in the first two positions of SEQ ID NO:8.

The invention can be carried out using any suitable biological sample obtained from a feline that would be expected to contain evidence FCoV1 if the feline is infected. Suitable sources of biological samples include but are not limited to blood, serum, plasma, mucosal scrapings, excreted matter, tissue biopsies, and saliva. The invention is believed to be suitable for diagnosis of FCoV1 infection status for any type of feline animal.

### BRIEF DESCRIPTION OF THE FIGURE

FIG. 1 provides an alignment of a representative FCoV1 genomic RNA (SEQ ID NO:1) encoding a representative FCoV1 spike protein, and the amino acid sequence (SEQ ID NO:3) of the FCoV1 spike protein encoded by the +1 open reading frame of SEQ ID NO:1.

### DETAILED DESCRIPTION OF THE INVENTION

The present invention provides a method for determining whether a feline is infected with FIPV, FECV, or with neither FIPV nor FECV. The method comprises determining from a biological sample obtained or derived from a feline the presence or absence of a mutation in the S1/S2 site, which is Arg Arg Ser Arg Arg Ser (SEQ ID NO:5) and determining the presence or absence of a mutation in the S2′ site, which is Xaa Xaa Ser Xaa Xaa Glu Asp Leu Leu Phe (SEQ ID NO:8), where Xaa Xaa in the first two positions of SEQ ID NO:8 are basic amino acids. The second two Xaa's in SEQ ID NO:8 (positions 4 and 5 of SEQ ID NO:8) can be any amino acid. In one embodiment, the amino acids in positions 4 and 5 of SEQ ID NO:8 are Ala and Val, respectively; in another embodiment, they are Thr and Val, respectively. These or any other changes in positions 4 and 5 of SEQ ID NO:8 are not considered mutations to the S2′ site for purposes of determining infection with FIPV or FECV. Accordingly, reference to a mutation of SEQ ID NO:8 that can affect whether the FCoV1 is FIPV or FECV refers to a mutation that changes the first two amino acids of SEQ ID NO:8 such that the first two positions do not comprise a dibasic couplet.

The feline is identified as infected with FIPV if a mutation is present in the S1/S2 site, the S2′ site, or a combination thereof, and is identified as infected with FECV if non-mutated S1/S2 and S2′ sites are present. If no mutated or non-mutated S1/S2 and S2′ sites are present, it is considered the feline is not infected with FECV or FIPV. The S1/S2 site and the S2′ site are also referred to herein as the “furin cleavage site” and the “dibasic cleavage site,” respectively.

It will be apparent from the present disclosure that any change in FCoV1 genomic RNA that results in a change in the amino acid sequence in the furin cleavage site of the spike protein is indicative that the feline animal from which the viral sample was obtained is or was infected with FIPV. However, we demonstrate that analysis of the furin cleavage site alone is not sufficient to provide a diagnostic tool that is desirable for use in a clinical setting. This is because determining the absence of a mutation in the furin cleavage site alone (and assuming the presence of an FCoV1 spike protein with a non-mutated furin cleavage site) is not enough to conclude that the feline animal is or was infected with FECV. In this regard, the present invention discloses the unexpected finding that, in addition to analysis of the furin cleavage site, the dibasic cleavage site must also be analyzed to eliminate false diagnosis of infection with FECV, when in fact the feline is infected with FIPV. Thus, we have unexpectedly discovered that mutation of the dibasic cleavage site, (even with a non-mutated furin cleavage site in the same FCoV1 spike protein) is positively correlated with FIPV infection. At least one advantage of the present invention is accordingly a reduced risk of false negative diagnosis of FECV. The invention therefore provides novel and clinically useful diagnostic tools that can be used for testing diverse populations of feline animals.

SEQ ID NO:1 provides the sequence of a representative FCoV1 viral+RNA strand encoding an FCoV1 spike protein wherein both the S1/S2 and the S2′ mutations are absent. These are considered “intact” S1/S2 and S2′ sites. SEQ ID NO:2 provides the sequence of the DNA equivalent of the viral RNA+strand shown in SEQ ID NO:1. Thus, SEQ ID NO:2 provides the sequence of a representative viral RNA+strand where each Uracil (U) is replaced by Thymine (T).

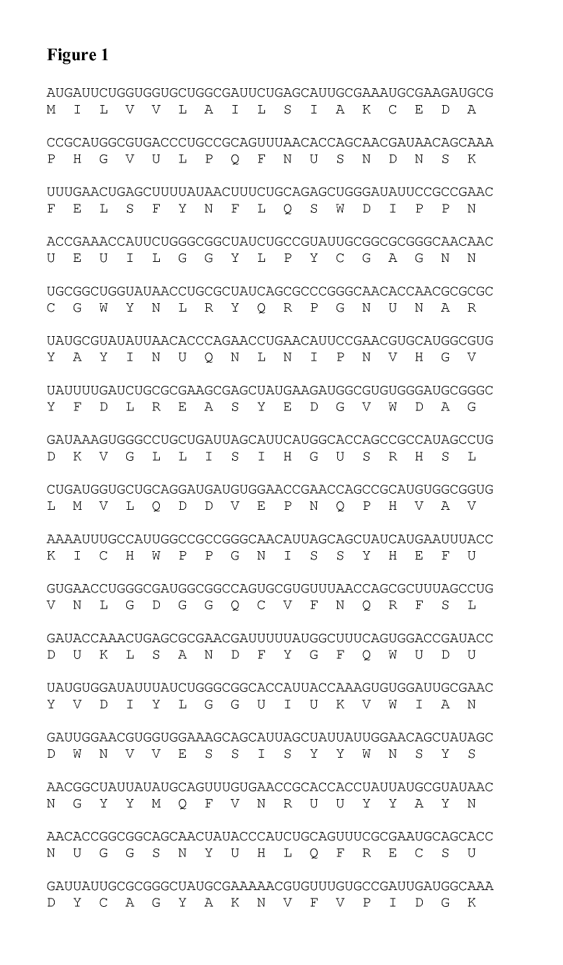

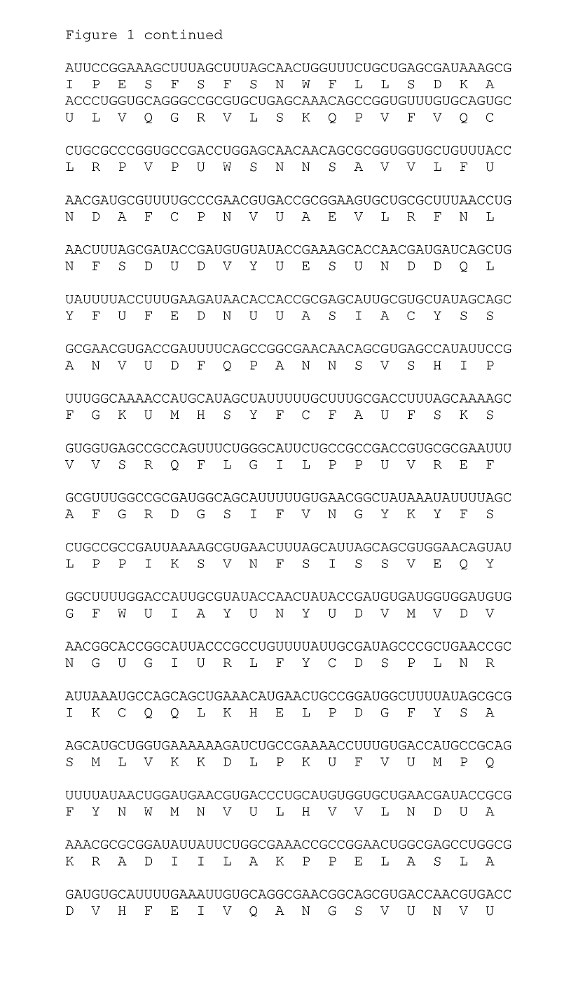

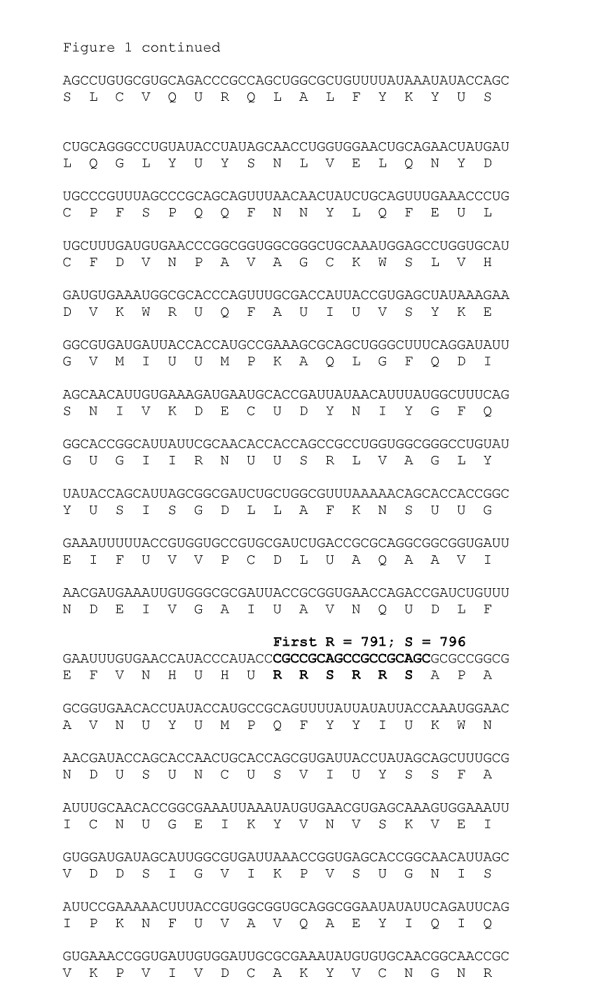

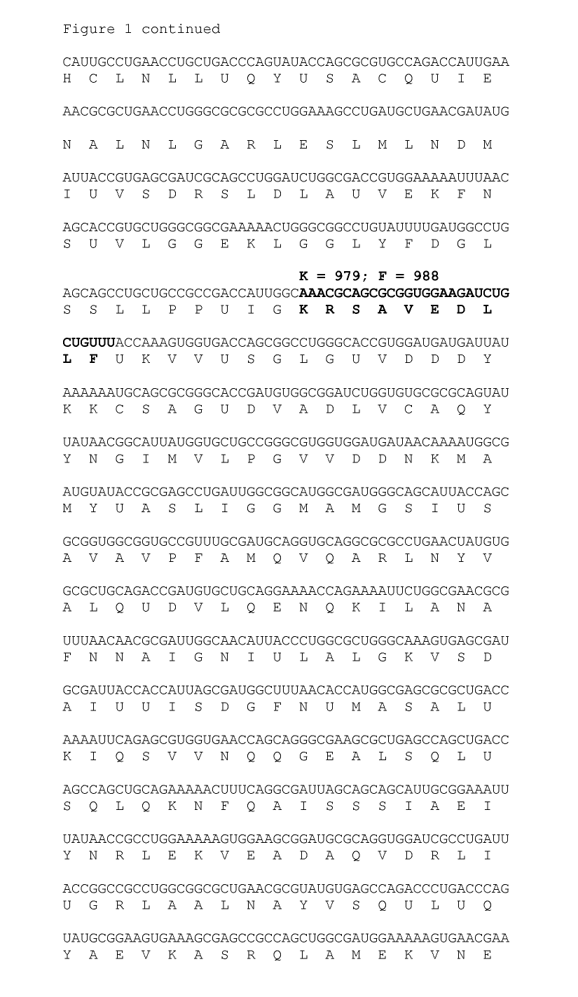

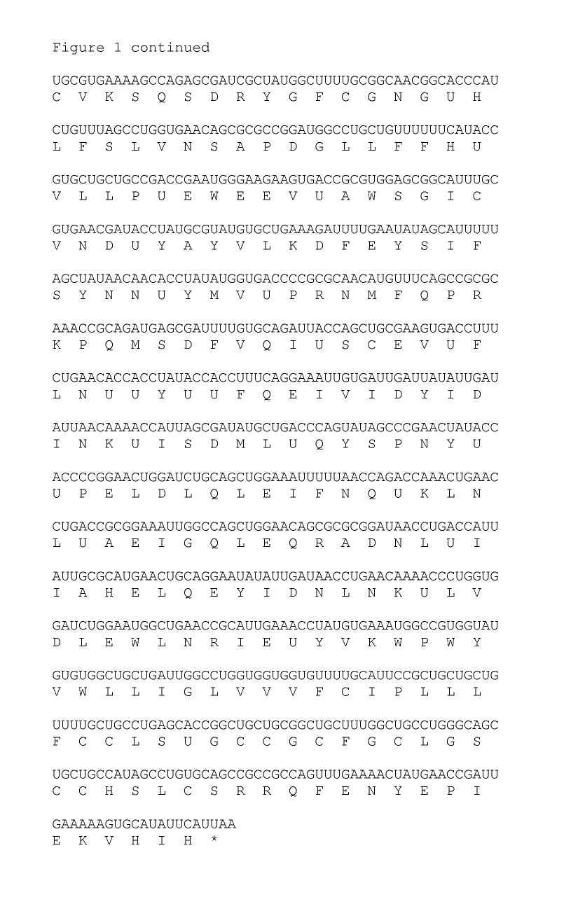

SEQ ID NO:3 provides the amino acid sequence of a representative FCoV1 viral spike protein encoded by the RNA sequence of SEQ ID NO:1. FIG. 1 provides an alignment of SEQ ID NO:1 and the amino acid sequence of the spike protein (SEQ ID NO:3) encoded by the +1 open reading frame of SEQ ID NO:1. In FIG. 1, the nucleotide sequence encoding an intact S1/S2 site (CGCCGCAGCCGCCGCAGC; SEQ ID NO:4) and an intact S2′ site are shown shaded and in bold. The amino acid sequences of the intact S1/S2 site (Arg Arg Ser Arg Arg Ser; SEQ ID NO:5; shown at positions 791-796 of SEQ ID NO:3) and that of an intact S2′ prime site (Lys Arg) at positions 979 and 980 in SEQ ID NO:3, are also shown as shaded and in bold. With respect to the S2′ site, we have determined that the sequence of six of the eight amino acid positions immediately C-terminal to the S2′ dibasic site are conserved throughout all FIPV and FECV samples analyzed to date. Thus, in one embodiment, the S2′ site is encoded by a nucleotide sequence which encodes the amino acid sequence Lys Arg Ser Ala Val Glu Asp Leu Leu Phe (SEQ ID NO:7). With respect to a mutation in the S2′ site, where the mutation of one or both Xaa in the first two positions of SEQ ID NO:8 is a change to a non-basic amino acid, it is considered that because the sequence of six of eight amino acids that are C-terminal to the Xaa Xaa are so highly conserved, determining a change in one or both Xaa amino acids such that the dibasic cleavage site is no longer present is equivalent to determining the entire SEQ ID NO:8, even if the eight C-terminal amino acids are not actively determined in any particular assay.

One representative sequence that encodes SEQ ID NO:7 is AAACGCAGCGCGGUGGAAGAUCUGCUGUUU (SEQ ID NO:6). (SEQ ID NO:6 and SEQ ID NO:7 are also shown as shaded and bold in FIG. 1). While SEQ ID NO:7 is a representative embodiment of an intact S2′ site in the spike protein and includes the conserved amino acids that are C-terminal to the KR sequence, those skilled in the art will recognize that other dibasic amino sequences may be substituted for the KR as reflected by SEQ ID NO:8, which permits any basic amino acid in its first two positions. Basic amino acids are those that are polar and positively charged at pH values below their pKa values, and are considered to be very hydrophilic. Basic amino acids that can be present in either or both and in any combination of the first two positions of SEQ ID NO:8 include Lys, Arg and His.

It will be recognized by those skilled in the art that, due to the redundancy in the genetic code, there are a plurality of nucleotide sequences that encode intact S1/S2 and S2′ sites. The present invention includes determining all of these nucleotide sequences as they may occur in various samples in order to determine that there is no mutation in either one or both sites. Further, the invention includes determining all nucleotide sequences that encode mutations in the amino acid sequences of the S1/S2 and the S2′ site. In connection with the S2′ site, the invention includes determining mutations to nucleotides that alter the Xaa Xaa in positions 1 and 2 of SEQ ID NO:8, such that the Xaa Xaa is no longer a dibasic amino acid couplet, whether or not such changes to the nucleotide sequences encoding the remaining C-terminal amino acids of SEQ ID NO:8 are also determined. In one embodiment, a mutation to the S2′ site results in a monobasic site.

It will be apparent from the foregoing that, in one embodiment, the invention provides for determining the presence of an S1/S2 mutation by determining any change in an FCoV1 nucleotide sequence that alters the S1/S2 site sequence of Arg Arg Ser Arg Arg Ser (SEQ ID NO:5). Likewise, the invention provides for determining the absence of a mutation to the S1/S2 site by determining the presence of any FCoV1 nucleotide sequence that encodes SEQ ID NO:5. Similarly, the invention provides for determining the presence of an S2′ mutation by determining any change in an FCoV1 nucleotide sequence that alters the first two amino acids of SEQ ID NO:8 such that a dibasic sequence is no longer comprised by those two positions. Accordingly, the invention also provides for determining the absence of an S2′ mutation by determining the presence of an FCoV1 nucleotide sequence encoding dibasic amino acids in the first two positions of SEQ ID NO:8. In an additional embodiment, the invention provides for determining that a feline animal is not infected with FECV or FIPV if no FCoV1 nucleotide sequences encoding SEQ ID NO:5 or a mutation thereto, and no FCoV1 nucleotide sequences encoding SEQ ID NO:7 or a mutation thereto, are determined from the sample obtained from the feline animal.

Those skilled in the art will recognize that SEQ ID NO:5, SEQ ID NO:7 and SEQ ID NO:8 refer to particular locations in FCoV1 spike protein (a representative amino acid sequence for which is provided in SEQ ID NO:3), but the spike protein of FCoVs can vary from virus to virus in amino acid content and length. For example, insertions, deletions and conservative and non-conservative amino acid substitutions can occur in portions of the protein that do not include the S1/S2 and S2′ sites. Thus the relative location of the S1/S2 and S2′ sites in terms of spike amino acid and nucleotide sequence and numbering can vary. It is expected that any such variations or combinations thereof in any particular FCoV1 will not affect the basic operation of the present invention, which encompasses all such variations in FCoV1 spike proteins and the nucleotide sequences encoding them. Further, given the benefit of the present disclosure, those skilled in the art will recognize where the S1/S2 and S2′ sites occur in any particular FCoV1 spike protein amino acid sequence, and accordingly where the nucleotide sequences encoding those mutations occur in any FCoV1 viral genome sequence, as well as in any nucleic acids amplified or otherwise derived therefrom. The invention also encompasses all nucleotide sequences (whether FCoV1 genomic RNA, its reverse complement and DNA equivalents) that encode all such FCoV1 spike proteins. Fragments of FCoV1 spike proteins and polynucleotides encoding them are also included within the scope of the invention.

Determining the presence or absence of the S1/S2 mutation and the S2′ mutation can be performed using any suitable techniques, reagents and compositions. Since the FECV and FIPV comprise positive-sense single-stranded RNA genomes, the invention includes but is not necessarily limited to detecting the presence or absence of mutations by direct analysis of viral RNA, by analysis of polynucleotides amplified from the viral RNA, by analysis of protein encoded by the viral RNA, and by any combination(s) thereof. Those skilled in the art will recognize that any technique now known or hereafter developed for detecting the mutations can be used to determine the presence or absence of the S1/S2 and S2′ sites or mutations thereof in polynucleotide sequences and/or amino acid sequences.

Suitable techniques for determining the presence or absence of FCoV1 mutations in nucleic acids include but are not limited to restriction enzyme mapping, hybridization of viral RNA or nucleic acids amplified therefrom to allele-specific probes or oligonucleotide arrays, by using various chip technologies, by polynucleotide sequencing techniques, and combinations thereof. Viral nucleic acids may be used directly or may be amplified enzymatically in vitro by, for example, use of the polymerase chain reaction (PCR) or any other in vitro amplification methods, which include but are not limited to ligase chain reactions (LCR). For amplification and/or sequencing reactions, primers can be designed which hybridize to a target sequence in the viral RNA and used for sequencing or for amplification to obtain nucleic acid amplification products (amplicons) which contain the S1/S2 mutation and/or the S2′ mutation site. Those skilled in the art will recognize how to design suitable primers and perform amplification reactions in order to carry out various embodiments of the method of the invention. In general, the primers should be long enough to be useful in sequencing and/or amplification reactions, and generally primers which are at least twelve bases in length are considered suitable for such purposes. It will be recognized by those skilled in the art that while particular sequences of primers are provided herein, other primer sequences can be designed to detect the presence or absence of the mutations. In this regard, amplification primers can be designed so that they amplify nucleotide sequences encoding one or both of the S1/S2 and S2′ sites.

In one embodiment, representative primers for use in amplification of FCoV1 spike protein-encoding nucleic acids include but are not limited to primers with the following sequences: 700Fw-5′TAT GGG TTT TGG ACC ATA GC 3′ (SEQ ID NO:9) and 702Rv 5′GCC ATT GTA ATA TTG GGC AC 3′ (SEQ ID NO:10).

In one embodiment, a polynucleotide sequence that encodes SEQ ID NO:5 or a change in the amino acid sequence of SEQ ID NO:5 is analyzed to determine the FCoV1 infection status of a feline. Accordingly, any length FCoV1 nucleotide sequence that comprises or consists of a sequence that encodes SEQ ID NO:5 or a mutation of SEQ ID NO:5 can be analyzed.

In one embodiment, to determine the FCoV1 infection status of a feline, a polynucleotide sequence that encodes SEQ ID NO:8 or a change in the Xaa Xaa amino acid sequence in the first two positions of SEQ ID NO:8 such that the dibasic couplet is not present in SEQ ID NO:8 is analyzed in performing the method of the invention. The sequence of any length FCoV1 nucleotide sequence that comprises or consists of a sequence that encodes SEQ ID NO:8 or a mutation to the dibasic sequence of SEQ ID NO:8 can be analyzed.

In one embodiment, a polynucleotide sequence encoding the amino acid sequence that comprises or consists of amino acid number 791-980 or 791-988, inclusive, of SEQ ID NO:3 can be analyzed to determine the presence or absence of one or both of the S1/S2 and S2′ sites.

In one embodiment of the invention, a polynucleotide that comprises from nucleotides 2371-2490, inclusive, in SEQ ID NO:1 or its DNA equivalent is amplified for use in detecting the presence or absence of the mutations. In another embodiment, a polynucleotide sequence that comprises from nucleotides 2371-2964, inclusive, in SEQ ID NO:1 or its DNA equivalent is amplified for use in the detecting the presence or absence of the mutations.

In certain embodiments, the invention provides a composition comprising an isolated FCoV1 viral polynucleotide and components used for nucleic acid hybridization and/or amplification. Accordingly the compositions can additionally comprise a DNA polymerase, a reverse transcriptase, an RNA-dependent-RNA-polmerase (RDRP), free nucleotide triphosphates, salts, buffers, other reagents typically employed to hybridize and/or amplify nucleic acids, and combinations of the foregoing. In one embodiment, the invention provides an isolated FCoV1 polynucleotide and/or a polynuceotide amplified from an FCoV1 polynucleotide, wherein the polynucleotide and/or the amplified polynucleotide is hybridized to one or more amplification or sequencing primers. The hybridized nucleic acids may be present in a buffer suitable for nucleic acid polymerization or sequencing reactions.

The invention also provides an isolated viral polynucleotide and/or a polynuceotide amplified from a viral polynucleotide wherein the polynucleotide is hybridized to at least one probe that is present in an array. The array may be present on, for example, a chip used to determine the presence or absence of a plurality of distinct polynucleotides. Such chips are commercially available and can be customized to detect the presence or absence of essentially any polynucleotide.

The present invention also includes determining the presence or absence of the S1/S2 mutation and/or the S2′ mutation by detecting all or a portion of a FCoV1 spike protein that contains the site where S1/S2 mutations and/or the S2′ mutations occur, or by detecting fragments of the spike protein that have been cleaved at one or both of the mutation sites. The presence or absence of the mutation(s) in the proteins can be determined using any suitable technique. For example, it is expected antibodies that are specific for the normal FCoV1 spike protein, and antibodies that are specific for a FCoV1 spike protein that contains the S1/S2 mutation and/or the S2′ mutation, or has been cleaved at either or both positions can be raised.

In various embodiments, the antibodies can recognize and discriminate between FECV and FIPV based on the presence or absence of one or both of the S1/S2 and S2′ mutations. It is expected that such antibodies can be raised using standard and well recognized techniques for immunization of mammals and production of antibody-producing hybridomas from the immunizations. It is also expected that the antibodies can be raised using any of a variety of compositions that contain the FCoV1 spike protein or fragments thereof as an immunogen. For example, the spike proteins can be present in purified whole FCoV1 viral particles or FCoV1 viral lysates, or lysates from infected cells, whether the cells are obtained from infected animals and/or are cultured in vitro. Isolated and/or purified spike protein or fragments thereof, and combinations thereof can also be used. Any of these compositions can be naturally occurring virus or virus components or fragments thereof, or they can be made recombinantly. Any of the foregoing compositions can comprise a normal spike protein amino acid sequence (i.e., a spike protein amino acid sequence that does not comprise either the furin mutation or the dibasic mutation). The compositions can also comprise a spike protein having an amino acid sequence that comprises one or both of the mutations. In the case of cleaved proteins or fragments designed to represent cleaved proteins, the cleaved portions or the fragments can comprise the portion of the cleaved site that remains in the cleavage products or that is present in the fragment that is designed to represent the cleaved protein. All combinations of these proteins and/or protein fragments for use in generating antibodies are encompassed within the scope of the invention.

In one embodiment, it is considered that a polypeptide comprising or consisting of the sequence of amino acid number 791-980 or 791-988, inclusive, of SEQ ID NO:3 can be used to generate antibodies that specifically recognize FCoV1 comprising intact S1/S2 and S2′ sites. Fragments of these polypeptides are also included. In alternative embodiments, it is contemplated that peptides that comprise or consist of SEQ ID NO:5, or peptides that comprise a change in the amino acid sequence of SEQ ID NO:5 can be used to create antibodies that can be used for performing the method of the invention. In other embodiments, peptides that comprise SEQ ID NO:7, or peptides that comprise a change in the one or both of the first two amino acids of sequence of SEQ ID NO:7 can be used to make antibodies that can be used for performing the method of the invention.

In one embodiment, one or more antibodies that can specifically recognize normal FCoV1 spike protein or FCoV1 spike protein having one or both mutations, or combinations thereof, can be provided in physical association with a solid matrix. The solid matrix may be present in a multi-well assay plate, beads, a lateral flow device or strip, or any other form or format that is suitable for keeping the antibody in a position whereby viral proteins present in or otherwise derived from a biological sample obtained from a feline can be captured and be detected. The antibodies may be covalently or non-covalently associated with the solid matrix. The assay may be configured to provide a detectable signal indicating the presence of normal spike protein, or spike protein comprising one or both mutations. Such immunodetection systems can be combined with suitable controls that can also be made with the FCoV1 proteins and peptides as described herein.

Any technique, device, system and/or reagents can be used to detect the different spike proteins in the biological sample. In non-limiting examples, the spike proteins can be detected and discriminated from one another using any immunodection techniques, which include but are not necessarily limited to Western blot, an enzyme-linked immunosorbent assay (ELISA), or any modification of such assays that are suitable for detecting proteins of interest.

In one embodiment, the invention provides an isolated and/or recombinant FCoV1 virus or FCoV1 virus spike protein with intact S1/S2 and S2′ sites that is present in a complex with an antibody that can discriminate such spike protein from a spike protein comprising a mutation of the S1/S2 and or the S2 site.

In one embodiment, the determination of the presence or absence of the mutation of the SEQ ID NO:5 and/or the determination of the presence or the absence of the mutation of the SEQ ID NO:8 is performed by determining a FCoV1 polypeptide sequence from the biological sample, wherein the FCoV1 polypeptide sequence is set forth in the SEQ ID NO:5 or the mutation of the SEQ ID NO:5, or the FCoV1 polypeptide sequence is set forth in the SEQ ID NO:8 or the mutation of one or both amino acids in the first two positions of SEQ ID NO:8 such that those positions no longer comprise a dibasic couplet.

The FCoV1 polypeptide sequence to be analyzed can be any suitable FCoV1 polypeptide that contains the amino acid sequence set forth in SEQ ID NO:5, SEQ ID NO:8 and/or a mutation thereof. In one example, the FCoV1 polypeptide sequence to be analyzed comprises an intact type 1 FCoV1 spike protein. In another example, the FCoV1 polypeptide sequence to be analyzed comprises a fragment of the type 1 FCoV1 spike protein, said fragment comprising a FCoV1 polypeptide sequence set forth in the SEQ ID NO:5 or the mutation of the SEQ ID NO:5, or said fragment comprising a FCoV1 polypeptide sequence set forth in the SEQ ID NO:8 or the mutation of the SEQ ID NO:8. The FCoV1 polypeptide sequence can be analyzed by any suitable methods. For example, the FCoV1 polypeptide sequence can be determined by an immunoassay, protein sequencing, mass spectrometry, or any other suitable methods.

In various embodiments, the invention further comprises fixing in a tangible medium the determination of whether or not a feline animal has been infected by FECV or FIPV. The tangible medium can be any type of tangible medium, such as any type of digital medium, including but not limited a DVD, a CD-ROM, a portable flash memory device, etc. The invention includes providing the tangible medium to a health care provider to develop a recommendation for treatment of infected feline animals.

Also provided in the present invention are kits for detecting the presence or absence of the mutations. The kits comprise reagents for nucleic acid based detection of the presence or absence of the mutation(s), or antibodies for detecting the presence or absence of the mutation(s) in the spike protein. In one embodiment, the kits comprise reagents for extraction/preparation of nucleic acid samples and pair(s) of specific primers for use in identification the mutations. In another embodiment, the kits provide antibodies and compositions used for probing samples with the antibodies to determine whether or not FECV or FIPV or neither virus is present in a biological sample.

In another aspect, the present invention provides for a composition for determining whether a feline is infected with FIPV or FECV comprising: a) a polynucleotide sequence for determining the presence or absence of a mutation in SEQ ID NO:5 (Arg Arg Ser Arg Arg Ser) in a FCoV1 spike protein; and b) a polynucleotide sequence for determining the presence or absence of the mutation in the dibasic couplet of SEQ ID NO:8 in the type 1 FCoV1 spike protein, wherein Xaa in the first two positions is a basic amino acid and wherein the mutation is a change to a non-basic amino acid in the first, second or both Xaa in the first two positions of SEQ ID NO:8.

In one embodiment, the composition comprises a pair of polynucleotide sequences for determining the presence or absence of a mutation in SEQ ID NO:5 (Arg Arg Ser Arg Arg Ser) in a type 1 feline coronavirus (FCoV1) spike protein. In another embodiment, the composition comprises a pair of polynucleotide sequences for determining the presence or absence of a mutation of SEQ ID NO:8 in the type 1 FCoV1 spike protein, wherein Xaa in the first two positions of SEQ ID NO:8 is a basic amino acid and wherein the mutation is a change to a non-basic amino acid in the first, second or both Xaa in the first two positions of SEQ ID NO:8.

The polynucleotide sequences can be, generally, substantially complementary to polynucleotide sequences that encode the amino acid sequence set forth in SEQ ID NO:5 or SEQ ID NO:8, or a mutation or a portion thereof. The polynucleotide sequences can also be, generally, substantially complementary to polynucleotide sequences that flank or are adjacent to the target polynucleotide sequences that encode the amino acid sequence set forth in SEQ ID NO:5 or SEQ ID NO:8, or a mutation or a portion thereof. In some embodiments, the polynucleotide sequences can hybridize to polynucleotide sequences that encode the amino acid sequence set forth in SEQ ID NO:5 or SEQ ID NO:8, or a mutation or a portion thereof, or polynucleotide sequences that flank or are adjacent to the target polynucleotide sequences that encode the amino acid sequence set forth in SEQ ID NO:5 or SEQ ID NO:8, or a mutation or a portion thereof, under low, middle or high stringent conditions. In some embodiments, the polynucleotide sequences are completely complementary to polynucleotide sequences that encode the amino acid sequence set forth in SEQ ID NO:5 or SEQ ID NO:8, or a mutation or a portion thereof, or polynucleotide sequences that flank or are adjacent to the target polynucleotide sequences that encode the amino acid sequence set forth in SEQ ID NO:5 or SEQ ID NO:8, or a mutation or a portion thereof.

As used herein, “stringency” of nucleic acid hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Current Protocols in Molecular Biology (Ausubel et al. eds., Wiley Interscience Publishers, 1995); Molecular Cloning: A Laboratory Manual (J. Sambrook, E. Fritsch, T. Maniatis eds., Cold Spring Harbor Laboratory Press, 2d ed. 1989); Wood et al., Proc. Natl. Acad. Sci. USA, 82:1585-1588 (1985).

The polynucleotide sequences can be used in any suitable form. For example, the polynucleotide sequences can be probes that are used in hybridization assays in determining the presence or absence of a mutation in SEQ ID NO:5 and/or SEQ ID NO:8 in a type 1 feline coronavirus (FCoV1) spike protein. In another example, the polynucleotide sequences can be primers that are used in amplifying and/or detecting a target polynucleotide sequence that may contain a mutation in SEQ ID NO:5 and/or SEQ ID NO:8 in a type 1 feline coronavirus (FCoV1) spike protein. In still another example, the polynucleotide sequence for determining the presence or absence of a mutation in SEQ ID NO:5 (Arg Arg Ser Arg Arg Ser) in FCoV1 spike protein and/or the polynucleotide sequence for determining the presence or absence of a mutation of SEQ ID NO: in the type 1 FCoV1 spike protein is immobilized on a solid surface or linked to a detectable label.

In still another aspect, the present invention provides for a kit for determining whether a feline is infected with FIPV or FECV) in a single container or separate containers, comprising: a) a polynucleotide sequence for determining the presence or absence of a mutation in SEQ ID NO:5 (Arg Arg Ser Arg Arg Ser) in a FCoV1 spike protein; and b) a polynucleotide sequence for determining the presence or absence of a mutation of SEQ ID NO:8 in the type 1 FCoV1 spike protein, wherein Xaa in the first two positions is a basic amino acid and wherein the mutation is a change to a non-basic amino acid in the first, second or both Xaa in the first two positions of SEQ ID NO:8. The kit can contain additional suitable components. For example, the kit can further comprise: a) a means for collecting a biological sample from a feline; b) a means for isolating, purifying and/or amplifying a FCoV1 polynucleotide sequence comprising a polynucleotide sequence set forth in SEQ ID NO:5 (Arg Arg Ser Arg Arg Ser) and/or SEQ ID NO:8; and/or c) an instruction for using the kit.

In yet another aspect, the present invention provides for a composition for determining whether a feline is infected with FIPV or FECV comprising: a) a specific binder that can distinguish a FCoV1 polypeptide comprising an amino acid sequence set forth in SEQ ID NO:5 (Arg Arg Ser Arg Arg Ser) from a FCoV1 polypeptide comprising a mutation in the amino acid sequence set forth in SEQ ID NO:5; and b) a specific binder that can distinguish a FCoV1 polypeptide comprising an amino acid sequence set forth in SEQ ID NO:8 from a FCoV1 polypeptide comprising a mutation in the amino acid residue(s) Xaa and/or Xaa set forth in the first two positions of SEQ ID NO:8.

Any suitable specific binders, e.g., antibodies, natural or engineered ligands that bind to the FCoV1 spike protein, aptamers, and small molecule binders, etc., can be used. As used herein, a “specific binder” refers to any substance that binds to target or analyte with desired affinity and/or specificity. In one embodiment, one or both specific binders are antibodies. As used herein, “antibody” includes not only intact polyclonal or monoclonal antibodies, but also fragments thereof (such as Fab, Fab′, F(ab′)₂, Fv), single chain (ScFv), a diabody, a multi-specific antibody formed from antibody fragments, mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class.

The specific binders can be used in any suitable form. For example, one or both specific binders can be immobilized on a solid surface or linked to a detectable label.

In yet another aspect, the present invention provides for a kit for determining whether a feline is infected with FIPV or FECV, in a single container or separate containers, comprising: a) a specific binder that can distinguish a FCoV1 polypeptide comprising an amino acid sequence set forth in SEQ ID NO:5 (Arg Arg Ser Arg Arg Ser) from a FCoV1 polypeptide comprising a mutation in the amino acid sequence set forth in SEQ ID NO:5; and b) a specific binder that can distinguish a FCoV1 polypeptide comprising an amino acid sequence set forth in SEQ ID NO:8 from a FCoV1 polypeptide comprising a mutation in the amino acid residue(s) Xaa and/or Xaa set forth in the first two positions of SEQ ID NO:8. The kit can contain additional suitable components. For example, the kit can further comprise: a) a means for collecting a biological sample from a feline; b) a means for isolating and/or purifying the FCoV1 polypeptide sequence comprising a polypeptide sequence set forth in SEQ ID NO:5 (Arg Arg Ser Arg Arg Ser) and/or SEQ ID NO:8 (Xaa Xaa Ser Xaa Xaa Glu Asp Leu Leu Phe); and/or c) an instruction for using the kit.

The invention can be carried out using any suitable biological sample obtained from a feline. A suitable biological sample is one that, if the feline was infected, would be expected to contain FCoV1 particles, nucleic acids encoding FCoV1 spike protein or portions thereof, or combinations thereof. The FCoV1 can be present in infected cells or in biological fluids. Suitable sources of biological sample include but are not limited to blood, serum, plasma, mucosal scrapings, excreted matter, tissue biopsies, and saliva. In one embodiment, the biological sample is blood. In other non-limiting embodiments, the biological sample is selected from feces, or biopsies from omentum, liver, jejunum, spleen, mesentery, kidney, mesenteric lymph node, or lower gut.

In one embodiment, the biological sample is obtained from the feline and used directly in determining whether the feline is infected with FIPV or FECV. In another embodiment, the biological sample is obtained from the feline and subjected to a processing step before the biological sample is used in determining whether the feline is infected with FIPV or FECV. In some examples, the processing step can be carried out to isolate, purify and/or amplify the FCoV1 polynucleotide or FCoV1 polypeptide sequence to be analyzed in determining whether the feline is infected with FIPV or FECV.

It is contemplated that diagnosis of FCoV1 infection of any feline animal can be achieved using the present invention. Non-limiting examples of feline animals for which a diagnosis can be made include but are not necessarily limited to domestic house cats (Felis catus), feral cats, and other wild cats including bobcat, cougar, mountain lion, tiger, jaguar, leopard, puma, cheetah, and lion.

Data presented herein show that the mutations within the consensus protease processing sites(s) of the FCoV1 spike protein correlates with the conversion of FECV to FIPV. Without intending to be bound by any particular theory, it is considered that this conversion causes a change in the entry pathway of the virus which then allow escape of the virus from gut epithelial cells and into monocytes and macrophages.

A summary table of the sequencing of FECV and FIPV spike genes in the S1/S2 and S2′ sites of Type 1 feline coronaviruses supporting our discovery are presented in Table 1 below. To obtain these data, viral RNA was extracted from stool samples (FECV), post-mortem liver samples (FIPV from clinical cases) or from infected cell lysates (FIPV from biological repositories), amplified by RT-PCR and the central portion of the spike gene sequenced.

TABLE 1

| | Source | | | | | |
|---|---|---|---|---|---|---|
| Status | Tissue | Name | S1/S2 | | S2′ | |
| FECV | Feces | CUA001 | THTRRSRRSAPA | SEQ ID NO: 11 | PTIGKRSAVED | SEQ ID NO: 12 |
| FECV | Feces | CUA002 | THTRRSRRSAPA | SEQ ID NO: 11 | PTIGKRSAVED | SEQ ID NO: 12 |
| FECV | Feces | CUA003 | THTRRSRRSAPA | SEQ ID NO: 11 | PTIGKRSAVED | SEQ ID NO: 12 |
| FECV | Feces | CUA004 | THTRRSRRSAPA | SEQ ID NO: 11 | PTIGKRSAVED | SEQ ID NO: 12 |
| FECV | Feces | CUA005 | THTRRSRRSAPA | SEQ ID NO: 11 | PTIGKRSAVED | SEQ ID NO: 12 |
| FECV | Feces | CUA006 | THTRRSRRSAPV | SEQ ID NO: 13 | PTIGKRSAVED | SEQ ID NO: 12 |
| FECV | Feces | CUA007 | THTRRSRRSAPI | SEQ ID NO: 14 | PPIGKRSAVED | SEQ ID NO: 15 |
| FECV | Feces | CUA008 | THTRRSRRSAPI | SEQ ID NO: 14 | PTIGKRSAVED | SEQ ID NO: 12 |
| FECV | Feces | CUA009 | THTRRSRRSAPI | SEQ ID NO: 14 | PTIGKRSAVED | SEQ ID NO: 12 |
| FECV | Feces | CUA010 | THTRRSRRSAPI | SEQ ID NO: 14 | PTIGKRSAVED | SEQ ID NO: 12 |
| FECV | Feces | CUA011 | THTRRSRRSAPA | SEQ ID NO: 11 | PTIGKRSAVED | SEQ ID NO: 12 |
| FECV | Feces | CUA012 | THTRRSRRSAPV | SEQ ID NO: 13 | PTIGKRSAVED | SEQ ID NO: 12 |
| FECV | Feces | CUA013 | THTRRSRRSAPA | SEQ ID NO: 11 | PTIGKRSAVED | SEQ ID NO: 12 |
| FECV | Feces | CUA014 | THTRRSRRSAPV | SEQ ID NO: 13 | PTIGKRSAVED | SEQ ID NO: 12 |
| FECV | Feces | CUA015 | THTRRSRRSAPV | SEQ ID NO: 13 | PTIGKRSAVED | SEQ ID NO: 12 |
| FECV | Feces | CUA016 | THTRRSRRSAPV | SEQ ID NO: 13 | PTIGKRSAVED | SEQ ID NO: 12 |
| FECV | Feces | CUA017 | THTRRSRRSAPV | SEQ ID NO: 13 | PTIGKRSAVED | SEQ ID NO: 12 |
| FECV | Feces | CUA018 | THTRRSRRSAPV | SEQ ID NO: 13 | PTIGKRSAVED | SEQ ID NO: 12 |
| FECV | Feces | CUA019 | THTRRSRRSAPV | SEQ ID NO: 13 | PTIGKRSAVED | SEQ ID NO: 12 |
| FECV | Feces | FECV-4594 | TQQRRSRRSTSD | SEQ ID NO: 16 | PTIGKRSAVED | SEQ ID NO: 12 |
| FECV | Feces | FECV-4582 | TQQRRSRRSTSD | SEQ ID NO: 16 | PTIGKRSAVED | SEQ ID NO: 12 |
| FECV | Feces | FECV-FCA4597 | TQQRRSRRSTSD | SEQ ID NO: 16 | PTIGKRSAVED | SEQ ID NO: 12 |
| FECV | Feces | FECV-FCA4606 | TQQRRSRRSTSD | SEQ ID NO: 16 | PTIGKRSAVED | SEQ ID NO: 12 |
| FECV | | UCD | THSRRSRRSTLT | SEQ ID NO: 17 | | |
| FECV | | RM | TQPRRSRRSTPN | SEQ ID NO: 18 | PTIGKRSAVED | SEQ ID NO: 12 |
| FIPV | | FIPV-4618 | TSSRSARRSTGE | SEQ ID NO: 19 | PTIGKSSVVED | SEQ ID NO: 20 |
| FIPV | Feces | FIPV-4662 | TPQRRSRSSTSD | SEQ ID NO: 21 | PTIGKRSTVED | SEQ ID NO: 22 |
| FIPV | | FIPV-4663 | TPQRRSRTSTSD | SEQ ID NO: 23 | PTIGKRSTVED | SEQ ID NO: 22 |
| FIPV | Omentum | CU-B001 | TRPRRSRRSTSE | SEQ ID NO: 24 | PRVGQRSAVED | SEQ ID NO: 25 |
| FIPV | Omentum | CU-B002 | TRPRRSRRSTSE | SEQ ID NO: 24 | PHIGQRSAVED | SEQ ID NO: 26 |
| FIPV | Omentum | CU-B003 | TRPGRSRRSTSE | SEQ ID NO: 27 | PHIGQRSAVED | SEQ ID NO: 26 |
| FIPV | Omentum | CU-B004 | TSSKRSLRSTPA | SEQ ID NO: 28 | PIVGKRSAVED | SEQ ID NO: 29 |
| FIPV | Omentum | CU-B005 | TRPRRSRRSTSE | SEQ ID NO: 24 | PRIGQRSAVED | SEQ ID NO: 30 |
| FIPV | Omentum | CU-B006 | TRPRRSRTSTSE | SEQ ID NO: 31 | PRVGKRSAVED | SEQ ID NO: 32 |
| FIPV | Omentum | CU-B007 | TRPRRSRGSTSE | SEQ ID NO: 33 | PRVGKRSAVED | SEQ ID NO: 32 |
| FIPV | Omentum | CU-B008 | TQSKRSRRSTPA | SEQ ID NO: 34 | PIVGKRSAVED | SEQ ID NO: 29 |
| FIPV | Omentum | CU-B009 | TLSRRSRRLTSE | SEQ ID NO: 35 | PRVGMRSTVED | SEQ ID NO: 36 |
| FIPV | Omentum | CU-A020 | TSSRSSRRSTSE | SEQ ID NO: 37 | SRIGERSAVED | SEQ ID NO: 38 |
| FIPV | Omentum | CU-A021 | SQSRRSRSSTSE | SEQ ID NO: 39 | PRVGKRSAVED | SEQ ID NO: 32 |
| FIPV | | UCD1 | THSRRSRGSTST | SEQ ID NO: 40 | PRVGQRSAVED | SEQ ID NO: 25 |
| FIPV | Liver | Black | TQAKRSRRPTSH | SEQ ID NO: 41 | PKIGVRSAVED | SEQ ID NO: 42 |
| FIPV | | Q66951_9ALP | TQARRSRSSTPN | SEQ ID NO: 43 | PKIGKRSAVED | SEQ ID NO: 44 |
| | | C FIPV | | | | |
| FIPV | | Q8JVL1_9ALP | TQAKRSRRPTSH | SEQ ID NO: 41 | PKIGARSAVED | SEQ ID NO: 45 |
| | | C FIPV | | | | |
| FIPV | Omentum | UCD11a | TSARKSRRSVTE | SEQ ID NO: 46 | LRIGKRSAVED | SEQ ID NO: 47 |
| FIPV | Omentum | UCD11a-b | TSARKSRRSVTE | SEQ ID NO: 46 | LRIGKRSAVED | SEQ ID NO: 47 |
| FIPV | Omentum | UCD11b-2a | TSARKSRRSVTE | SEQ ID NO: 46 | LRIGKRSAVED | SEQ ID NO: 47 |
| FIPV | Omentum | UCD11b-2b | TSARKSRRSVTE | SEQ ID NO: 46 | LRIGKRSAVED | SEQ ID NO: 47 |
| FIPV | Omentum | UCD12 | TSARKSRRSVTE | SEQ ID NO: 46 | PRVGMRSAVED | SEQ ID NO: 48 |
| FIPV | Omentum | UCD13 | TSARKSLRSVTE | SEQ ID NO: 49 | PRVGKSSAVED | SEQ ID NO: 50 |
| FIPV | Omentum | UCD14 | TSARKSLRSVTE | SEQ ID NO: 49 | PRIGKRSAVED | SEQ ID NO: 51 |
| FIPV | Jejunum | FCoV1Je | TQPRQSRRSANP | SEQ ID NO: 52 | | |
| FIPV | Blood | CU-A022 | RRSRSS | SEQ ID NO: 53 | | |
| FIPV | Spleen | FFPE FIPV | HTSRRSRGSAPN | SEQ ID NO: 54 | | |
| | | 327-2 | | | | |
| FIPV | Mesentery | FFPE FIPV | HHSRRSRGSAPN | SEQ ID NO: 55 | | |
| | | 327-6 | | | | |
| FIPV | Mesentery | FFPE FIPV | TSSRRSPRSTLD | SEQ ID NO: 56 | | |
| | | 129308-Mes | | | | |
| FIPV | Kidney | FFPE FIPV | PQPRRARMSVPE | SEQ ID NO: 57 | | |
| | | 15390 | | | | |
| FIPV | Kidney | FFPE 77-3 | THSRRSRMSTQN | SEQ ID NO: 58 | | |
| FIPV | Cerebellum | FFPE 77-6 | THSRRSLRSTQN | SEQ ID NO: 59 | | |
| FIPV | Mesenteric | FFPE FIPV | TQSRRASTSTSN | SEQ ID NO: 60 | | |
| | Lymph | 244 MLN | | | | |
| | node | | | | | |
| FIPV | Mesentery | FFPE FIPV | HSRRRASTSTSN | SEQ ID NO: 61 | | |
| | | 244-MES | | | | |
| FIPV | Lower Gut | FFPE FIPV | TSSRRSLRSTVR | SEQ ID NO: 62 | | |
| | | 129308-LG | | | | |

It will be recognized from the data presented in Table 1 that the there is an invariant RRSRRS (SEQ ID NO:5) motif at the S1/S2 junction for all type 1 FECVs tested. In contrast, most type 1 FIPV spike proteins have a mutation in the RRSRRS (SEQ ID NO:5) motif. For those FIPVs with an intact RRSRRS (SEQ ID NO:5) motif, there is a mutation in the S2′ site, such that the dibasic KR motif is monobasic (i.e., a single R or K).

While the invention has been described through illustrative examples, routine modifications will be apparent to those skilled in the art, which modifications are intended to be within the scope of the invention.

We claim:

1. A composition for determining whether a feline is infected with pathogenic Feline Infectious Peritonitis Virus (FIPV) or Feline Enteric Infection Virus (FECV) comprising:
a) a polynucleotide sequence for determining the presence or absence of a mutation in SEQ ID NO:5 (Arg Arg Ser Arg Arg Ser) in a type 1 feline coronavirus (FCoV1) spike protein; and
b) a polynucleotide sequence for determining the presence or absence of a mutation of SEQ ID NO:8 (Xaa Xaa Ser Ala Val Glu Asp) in the type 1 FCoV1 spike protein, wherein Xaa is a basic amino acid and wherein the mutation is a change to a non-basic amino acid in the first, second or both Xaa in the SEQ ID NO:8.

2. The composition of claim 1, which comprises a pair of polynucleotide sequences for determining the presence or absence of a mutation in SEQ ID NO:5 (Arg Arg Ser Arg Arg Ser) in a type 1 feline coronavirus (FCoV1) spike protein.

3. The composition of claim 1, which comprises a pair of polynucleotide sequences for determining the presence or absence of a mutation of SEQ ID NO:8 (Xaa Xaa Ser Ala Val Glu Asp) in the type 1 FCoV1 spike protein, wherein Xaa is a basic amino acid and wherein the mutation is a change to a non-basic amino acid in the first, second or both Xaa in the SEQ ID NO:8.

4. The composition of claim 1, wherein the polynucleotide sequence for determining the presence or absence of a mutation in SEQ ID NO:5 (Arg Arg Ser Arg Arg Ser) in a type 1 feline coronavirus (FCoV1) spike protein and/or the polynucleotide sequence for determining the presence or absence of a mutation of SEQ ID NO:8 (Xaa Xaa Ser Ala Val Glu Asp) in the type 1 FCoV1 spike protein is immobilized on a solid surface or linked to a detectable label.

5. A kit for determining whether a feline is infected with pathogenic Feline Infectious Peritonitis Virus (FIPV) or Feline Enteric Infection Virus (FECV), in a single container or separate containers, comprising:
a) a polynucleotide sequence for determining the presence or absence of a mutation in SEQ ID NO:5 (Arg Arg Ser Arg Arg Ser) in a type 1 feline coronavirus (FCoV1) spike protein; and
b) a polynucleotide sequence for determining the presence or absence of a mutation of SEQ ID NO:8 (Xaa Xaa Ser Ala Val Glu Asp) in the type 1 FCoV1 spike protein, wherein Xaa is a basic amino acid and wherein the mutation is a change to a non-basic amino acid in the first, second or both Xaa in the SEQ ID NO:8.

6. The kit of claim 5, which further comprises:
a) a means for collecting a biological sample from a feline;
b) a means for isolating, purifying and/or amplifying a FCoV1 polynucleotide sequence comprising a polynucleotide sequence set forth in SEQ ID NO:5 (Arg Arg Ser Arg Arg Ser) and/or SEQ ID NO:8 (Xaa Xaa Ser Ala Val Glu Asp); and/or
c) an instruction for using the kit.

7. A composition for determining whether a feline is infected with pathogenic Feline Infectious Peritonitis Virus (FIPV) or Feline Enteric Infection Virus (FECV) comprising:
a) a specific binder that can distinguish a FCoV1 polypeptide comprising an amino acid sequence set forth in SEQ ID NO:5 (Arg Arg Ser Arg Arg Ser) from a FCoV1 polypeptide comprising a mutation in the amino acid sequence set forth in SEQ ID NO:5; and
b) a specific binder that can distinguish a FCoV1 polypeptide comprising an amino acid sequence set forth in SEQ ID NO:8 (Xaa Xaa Ser Ala Val Glu Asp) from a FCoV1 polypeptide comprising a mutation in the amino acid residue(s) Xaa and/or Xaa set forth in SEQ ID NO:8.

8. The composition of claim 7, wherein one or both specific binders are antibodies.

9. The composition of claim 7, wherein one or both specific binders are immobilized on a solid surface or linked to a detectable label.


SEQUENCE LISTING (62 sequences)

SEQ ID NO: 1 - RNA, 4407 nt, feline coronavirus
augauucugguggugcuggcgauucugagcauugcgaaaugcgaagaugcgccgcauggcgugacccugccgcaguuuaacaccagcaacgauaacagcaaauuugaacugagcuuuuauaacuuucugcagagcugggauauuccgccgaacaccgaaaccauucugggcggcuaucugccguauugcggcgcgggcaacaacugcggcugguauaaccugcgcuaucagcgcccgggcaacaccaacgcgcgcuaugcguauauuaacacccagaaccugaacauuccgaacgugcauggcguguauuuugaucugcgcgaagcgagcuaugaagauggcgugugggaugcgggcgauaaagugggccugcugauuagcauucauggcaccagccgccauagccugcugauggugcugcaggaugauguggaaccgaaccagccgcauguggcggugaaaauuugccauuggccgccgggcaacauuagcagcuaucaugaauuuaccgugaaccugggcgauggcggccagugcguguuuaaccagcgcuuuagccuggauaccaaacugagcgcgaacgauuuuuauggcuuucaguggaccgauaccuauguggauauuuaucugggcggcaccauuaccaaaguguggauugcgaacgauuggaacgugguggaaagcagcauuagcuauuauuggaacagcuauagcaacggcuauuauaugcaguuugugaaccgcaccaccuauuaugcguauaacaacaccggcggcagcaacuauacccaucugcaguuucgcgaaugcagcaccgauuauugcgcgggcuaugcgaaaaacguguuugugccgauugauggcaaaauuccggaaagcuuuagcuuuagcaacugguuucugcugagcgauaaagcgacccuggugcagggccgcgugcugagcaaacagccgguguuugugcagugccugcgcccggugccgaccuggagcaacaacagcgcgguggugcuguuuaccaacgaugcguuuugcccgaacgugaccgcggaagugcugcgcuuuaaccugaacuuuagcgauaccgauguguauaccgaaagcaccaacgaugaucagcuguauuuuaccuuugaagauaacaccaccgcgagcauugcgugcuauagcagcgcgaacgugaccgauuuucagccggcgaacaacagcgugagccauauuccguuuggcaaaaccaugcauagcuauuuuugcuuugcgaccuuuagcaaaagcguggugagccgccaguuucugggcauucugccgccgaccgugcgcgaauuugcguuuggccgcgauggcagcauuuuugugaacggcuauaaauauuuuagccugccgccgauuaaaagcgugaacuuuagcauuagcagcguggaacaguauggcuuuuggaccauugcguauaccaacuauaccgaugugaugguggaugugaacggcaccggcauuacccgccuguuuuauugcgauagcccgcugaaccgcauuaaaugccagcagcugaaacaugaacugccggauggcuuuuauagcgcgagcaugcuggugaaaaaagaucugccgaaaaccuuugugaccaugccgcaguuuuauaacuggaugaacgugacccugcauguggugcugaacgauaccgcgaaacgcgcggauauuauucuggcgaaaccgccggaacuggcgagccuggcggaugugcauuuugaaauugugcaggcgaacggcagcgugaccaacgugaccagccugugcgugcagacccgccagcuggcgcuguuuuauaaauauaccagccugcagggccuguauaccuauagcaaccugguggaacugcagaacuaugauugcccguuuagcccgcagcaguuuaacaacuaucugcaguuugaaacccugugcuuugaugugaacccggcgguggcgggcugcaaauggagccuggugcaugaugugaaauggcgcacccaguuugcgaccauuaccgugagcuauaaagaaggcgugaugauuaccaccaugccgaaagcgcagcugggcuuucaggauauuagcaacauugugaaagaugaaugcaccgauuauaacauuuauggcuuucagggcaccggcauuauucgcaacaccaccagccgccugguggcgggccuguauuauaccagcauuagcggcgaucugcuggcguuuaaaaacagcaccaccggcgaaauuuuuaccguggugccgugcgaucugaccgcgcaggcggcggugauuaacgaugaaauugugggcgcgauuaccgcggugaaccagaccgaucuguuugaauuugugaaccauacccauacccgccgcagccgccgcagcgcgccggcggcggugaacaccuauaccaugccgcaguuuuauuauauuaccaaauggaacaacgauaccagcaccaacugcaccagcgugauuaccuauagcagcuuugcgauuugcaacaccggcgaaauuaaauaugugaacgugagcaaaguggaaauuguggaugauagcauuggcgugauuaaaccggugagcaccggcaacauuagcauuccgaaaaacuuuaccguggcggugcaggcggaauauauucagauucaggugaaaccggugauuguggauugcgcgaaauaugugugcaacggcaaccgccauugccugaaccugcugacccaguauaccagcgcgugccagaccauugaaaacgcgcugaaccugggcgcgcgccuggaaagccugaugcugaacgauaugauuaccgugagcgaucgcagccuggaucuggcgaccguggaaaaauuuaacagcaccgugcugggcggcgaaaaacugggcggccuguauuuugauggccugagcagccugcugccgccgaccauuggcaaacgcagcgcgguggaagaucugcuguuuaccaaaguggugaccagcggccugggcaccguggaugaugauuauaaaaaaugcagcgcgggcaccgauguggcggaucuggugugcgcgcaguauuauaacggcauuauggugcugccgggcgugguggaugauaacaaaauggcgauguauaccgcgagccugauuggcggcauggcgaugggcagcauuaccagcgcgguggcggugccguuugcgaugcaggugcaggcgcgccugaacuauguggcgcugcagaccgaugugcugcaggaaaaccagaaaauucuggcgaacgcguuuaacaacgcgauuggcaacauuacccuggcgcugggcaaagugagcgaugcgauuaccaccauuagcgauggcuuuaacaccauggcgagcgcgcugaccaaaauucagagcguggugaaccagcagggcgaagcgcugagccagcugaccagccagcugcagaaaaacuuucaggcgauuagcagcagcauugcggaaauuuauaaccgccuggaaaaaguggaagcggaugcgcagguggaucgccugauuaccggccgccuggcggcgcugaacgcguaugugagccagacccugacccaguaugcggaagugaaagcgagccgccagcuggcgauggaaaaagugaacgaaugcgugaaaagccagagcgaucgcuauggcuuuugcggcaacggcacccaucuguuuagccuggugaacagcgcgccggauggccugcuguuuuuucauaccgugcugcugccgaccgaaugggaagaagugaccgcguggagcggcauuugcgugaacgauaccuaugcguaugugcugaaagauuuugaauauagcauuuuuagcuauaacaacaccuauauggugaccccgcgcaacauguuucagccgcgcaaaccgcagaugagcgauuuugugcagauuaccagcugcgaagugaccuuucugaacaccaccuauaccaccuuucaggaaauugugauugauuauauugauauuaacaaaaccauuagcgauaugcugacccaguauagcccgaacuauaccaccccggaacuggaucugcagcuggaaauuuuuaaccagaccaaacugaaccugaccgcggaaauuggccagcuggaacagcgcgcggauaaccugaccauuauugcgcaugaacugcaggaauauauugauaaccugaacaaaacccugguggaucuggaauggcugaaccgcauugaaaccuaugugaaauggccgugguauguguggcugcugauuggccugguggugguguuuugcauuccgcugcugcuguuuugcugccugagcaccggcugcugcggcugcuuuggcugccugggcagcugcugccauagccugugcagccgccgccaguuugaaaacuaugaaccgauugaaaaagugcauauucauuaa

SEQ ID NO: 2 - DNA, 4407 bp, artificial sequence
  DNA equivalent of FCoV1 spike genomic RNA
atgattctggtggtgctggcgattctgagcattgcgaaatgcgaagatgcgccgcatggcgtgaccctgccgcagtttaacaccagcaacgataacagcaaatttgaactgagcttttataactttctgcagagctgggatattccgccgaacaccgaaaccattctgggcggctatctgccgtattgcggcgcgggcaacaactgcggctggtataacctgcgctatcagcgcccgggcaacaccaacgcgcgctatgcgtatattaacacccagaacctgaacattccgaacgtgcatggcgtgtattttgatctgcgcgaagcgagctatgaagatggcgtgtgggatgcgggcgataaagtgggcctgctgattagcattcatggcaccagccgccatagcctgctgatggtgctgcaggatgatgtggaaccgaaccagccgcatgtggcggtgaaaatttgccattggccgccgggcaacattagcagctatcatgaatttaccgtgaacctgggcgatggcggccagtgcgtgtttaaccagcgctttagcctggataccaaactgagcgcgaacgatttttatggctttcagtggaccgatacctatgtggatatttatctgggcggcaccattaccaaagtgtggattgcgaacgattggaacgtggtggaaagcagcattagctattattggaacagctatagcaacggctattatatgcagtttgtgaaccgcaccacctattatgcgtataacaacaccggcggcagcaactatacccatctgcagtttcgcgaatgcagcaccgattattgcgcgggctatgcgaaaaacgtgtttgtgccgattgatggcaaaattccggaaagctttagctttagcaactggtttctgctgagcgataaagcgaccctggtgcagggccgcgtgctgagcaaacagccggtgtttgtgcagtgcctgcgcccggtgccgacctggagcaacaacagcgcggtggtgctgtttaccaacgatgcgttttgcccgaacgtgaccgcggaagtgctgcgctttaacctgaactttagcgataccgatgtgtataccgaaagcaccaacgatgatcagctgtattttacctttgaagataacaccaccgcgagcattgcgtgctatagcagcgcgaacgtgaccgattttcagccggcgaacaacagcgtgagccatattccgtttggcaaaaccatgcatagctatttttgctttgcgacctttagcaaaagcgtggtgagccgccagtttctgggcattctgccgccgaccgtgcgcgaatttgcgtttggccgcgatggcagcatttttgtgaacggctataaatattttagcctgccgccgattaaaagcgtgaactttagcattagcagcgtggaacagtatggcttttggaccattgcgtataccaactataccgatgtgatggtggatgtgaacggcaccggcattacccgcctgttttattgcgatagcccgctgaaccgcattaaatgccagcagctgaaacatgaactgccggatggcttttatagcgcgagcatgctggtgaaaaaagatctgccgaaaacctttgtgaccatgccgcagttttataactggatgaacgtgaccctgcatgtggtgctgaacgataccgcgaaacgcgcggatattattctggcgaaaccgccggaactggcgagcctggcggatgtgcattttgaaattgtgcaggcgaacggcagcgtgaccaacgtgaccagcctgtgcgtgcagacccgccagctggcgctgttttataaatataccagcctgcagggcctgtatacctatagcaacctggtggaactgcagaactatgattgcccgtttagcccgcagcagtttaacaactatctgcagtttgaaaccctgtgctttgatgtgaacccggcggtggcgggctgcaaatggagcctggtgcatgatgtgaaatggcgcacccagtttgcgaccattaccgtgagctataaagaaggcgtgatgattaccaccatgccgaaagcgcagctgggctttcaggatattagcaacattgtgaaagatgaatgcaccgattataacatttatggctttcagggcaccggcattattcgcaacaccaccagccgcctggtggcgggcctgtattataccagcattagcggcgatctgctggcgtttaaaaacagcaccaccggcgaaatttttaccgtggtgccgtgcgatctgaccgcgcaggcggcggtgattaacgatgaaattgtgggcgcgattaccgcggtgaaccagaccgatctgtttgaatttgtgaaccatacccatacccgccgcagccgccgcagcgcgccggcggcggtgaacacctataccatgccgcagttttattatattaccaaatggaacaacgataccagcaccaactgcaccagcgtgattacctatagcagctttgcgatttgcaacaccggcgaaattaaatatgtgaacgtgagcaaagtggaaattgtggatgatagcattggcgtgattaaaccggtgagcaccggcaacattagcattccgaaaaactttaccgtggcggtgcaggcggaatatattcagattcaggtgaaaccggtgattgtggattgcgcgaaatatgtgtgcaacggcaaccgccattgcctgaacctgctgacccagtataccagcgcgtgccagaccattgaaaacgcgctgaacctgggcgcgcgcctggaaagcctgatgctgaacgatatgattaccgtgagcgatcgcagcctggatctggcgaccgtggaaaaatttaacagcaccgtgctgggcggcgaaaaactgggcggcctgtattttgatggcctgagcagcctgctgccgccgaccattggcaaacgcagcgcggtggaagatctgctgtttaccaaagtggtgaccagcggcctgggcaccgtggatgatgattataaaaaatgcagcgcgggcaccgatgtggcggatctggtgtgcgcgcagtattataacggcattatggtgctgccgggcgtggtggatgataacaaaatggcgatgtataccgcgagcctgattggcggcatggcgatgggcagcattaccagcgcggtggcggtgccgtttgcgatgcaggtgcaggcgcgcctgaactatgtggcgctgcagaccgatgtgctgcaggaaaaccagaaaattctggcgaacgcgtttaacaacgcgattggcaacattaccctggcgctgggcaaagtgagcgatgcgattaccaccattagcgatggctttaacaccatggcgagcgcgctgaccaaaattcagagcgtggtgaaccagcagggcgaagcgctgagccagctgaccagccagctgcagaaaaactttcaggcgattagcagcagcattgcggaaatttataaccgcctggaaaaagtggaagcggatgcgcaggtggatcgcctgattaccggccgcctggcggcgctgaacgcgtatgtgagccagaccctgacccagtatgcggaagtgaaagcgagccgccagctggcgatggaaaaagtgaacgaatgcgtgaaaagccagagcgatcgctatggcttttgcggcaacggcacccatctgtttagcctggtgaacagcgcgccggatggcctgctgttttttcataccgtgctgctgccgaccgaatgggaagaagtgaccgcgtggagcggcatttgcgtgaacgatacctatgcgtatgtgctgaaagattttgaatatagcatttttagctataacaacacctatatggtgaccccgcgcaacatgtttcagccgcgcaaaccgcagatgagcgattttgtgcagattaccagctgcgaagtgacctttctgaacaccacctataccacctttcaggaaattgtgattgattatattgatattaacaaaaccattagcgatatgctgacccagtatagcccgaactataccaccccggaactggatctgcagctggaaatttttaaccagaccaaactgaacctgaccgcggaaattggccagctggaacagcgcgcggataacctgaccattattgcgcatgaactgcaggaatatattgataacctgaacaaaaccctggtggatctggaatggctgaaccgcattgaaacctatgtgaaatggccgtggtatgtgtggctgctgattggcctggtggtggtgttttgcattccgctgctgctgttttgctgcctgagcaccggctgctgcggctgctttggctgcctgggcagctgctgccatagcctgtgcagccgccgccagtttgaaaactatgaaccgattgaaaaagtgcatattcattaa

SEQ ID NO: 3 - protein, 1468 aa, feline coronavirus
MILVVLAILSIAKCEDAPHGVTLPQFNTSNDNSKFELSFYNFLQSWDIPPNTETILGGYLPYCGAGNNCGWYNLRYQRPGNTNARYAYINTQNLNIPNVHGVYFDLREASYEDGVWDAGDKVGLLISIHGTSRHSLLMVLQDDVEPNQPHVAVKICHWPPGNISSYHEFTVNLGDGGQCVFNQRFSLDTKLSANDFYGFQWTDTYVDIYLGGTITKVWIANDWNVVESSISYYWNSYSNGYYMQFVNRTTYYAYNNTGGSNYTHLQFRECSTDYCAGYAKNVFVPIDGKIPESFSFSNWFLLSDKATLVQGRVLSKQPVFVQCLRPVPTWSNNSAVVLFTNDAFCPNVTAEVLRFNLNFSDTDVYTESTNDDQLYFTFEDNTTASIACYSSANVTDFQPANNSVSHIPFGKTMHSYFCFATFSKSVVSRQFLGILPPTVREFAFGRDGSIFVNGYKYFSLPPIKSVNFSISSVEQYGFWTIAYTNYTDVMVDVNGTGITRLFYCDSPLNRIKCQQLKHELPDGFYSASMLVKKDLPKTFVTMPQFYNWMNVTLHVVLNDTAKRADIILAKPPELASLADVHFEIVQANGSVTNVTSLCVQTRQLALFYKYTSLQGLYTYSNLVELQNYDCPFSPQQFNNYLQFETLCFDVNPAVAGCKWSLVHDVKWRTQFATITVSYKEGVMITTMPKAQLGFQDISNIVKDECTDYNIYGFQGTGIIRNTTSRLVAGLYYTSISGDLLAFKNSTTGEIFTVVPCDLTAQAAVINDEIVGAITAVNQTDLFEFVNHTHTRRSRRSAPAAVNTYTMPQFYYITKWNNDTSTNCTSVITYSSFAICNTGEIKYVNVSKVEIVDDSIGVIKPVSTGNISIPKNFTVAVQAEYIQIQVKPVIVDCAKYVCNGNRHCLNLLTQYTSACQTIENALNLGARLESLMLNDMITVSDRSLDLATVEKFNSTVLGGEKLGGLYFDGLSSLLPPTIGKRSAVEDLLFTKVVTSGLGTVDDDYKKCSAGTDVADLVCAQYYNGIMVLPGVVDDNKMAMYTASLIGGMAMGSITSAVAVPFAMQVQARLNYVALQTDVLQENQKILANAFNNAIGNITLALGKVSDAITTISDGFNTMASALTKIQSVVNQQGEALSQLTSQLQKNFQAISSSIAEIYNRLEKVEADAQVDRLITGRLAALNAYVSQTLTQYAEVKASRQLAMEKVNECVKSQSDRYGFCGNGTHLFSLVNSAPDGLLFFHTVLLPTEWEEVTAWSGICVNDTYAYVLKDFEYSIFSYNNTYMVTPRNMFQPRKPQMSDFVQITSCEVTFLNTTYTTFQEIVIDYIDINKTISDMLTQYSPNYTTPELDLQLEIFNQTKLNLTAEIGQLEQRADNLTIIAHELQEYIDNLNKTLVDLEWLNRIETYVKWPWYVWLLIGLVVVFCIPLLLFCCLSTGCCGCFGCLGSCCHSLCSRRQFENYEPIEKVHIH

SEQ ID NO: 4 - RNA, 18 nt, feline coronavirus
cgccgcagccgccgcagc

SEQ ID NO: 5 - protein, 6 aa, feline coronavirus
RRSRRS

SEQ ID NO: 6 - RNA, 30 nt, feline coronavirus
aaacgcagcgcgguggaagaucugcuguuu

SEQ ID NO: 7 - protein, 10 aa, feline coronavirus
KRSAVEDLLF

SEQ ID NO: 8 - protein, 10 aa, artificial sequence
  S2′ site showing variable position of dibasic site and variable amino acids is 4 and 5 position
XXSXXEDLLF

SEQ ID NO: 9 - DNA, 20 bp, artificial sequence
  forward primer for amplifying FCoV1 sites
tatgggttttggaccatagc

SEQ ID NO: 10 - DNA, 20 bp, artificial sequence
  reverse primer for amplification of FCoV1 sites
gccattgtaatattgggcac

SEQ ID NO: 11 - protein, 12 aa, feline coronavirus
THTRRSRRSAPA

SEQ ID NO: 12 - protein, 11 aa, feline coronavirus
PTIGKRSAVED

SEQ ID NO: 13 - protein, 12 aa, feline coronavirus
THTRRSRRSAPV

SEQ ID NO: 14 - protein, 12 aa, feline coronavirus
THTRRSRRSAPI

SEQ ID NO: 15 - protein, 11 aa, feline coronavirus
PPIGKRSAVED

SEQ ID NO: 16 - protein, 12 aa, feline coronavirus
TQQRRSRRSTSD

SEQ ID NO: 17 - protein, 12 aa, feline coronavirus
THSRRSRRSTLT

SEQ ID NO: 18 - protein, 12 aa, feline coronavirus
TQPRRSRRSTPN

SEQ ID NO: 19 - protein, 12 aa, feline coronavirus
TSSRSARRSTGE

SEQ ID NO: 20 - protein, 11 aa, feline coronavirus
PTIGKSSVVED

SEQ ID NO: 21 - protein, 12 aa, feline coronavirus
TPQRRSRSSTSD

SEQ ID NO: 22 - protein, 11 aa, feline coronavirus
PTIGKRSTVED

SEQ ID NO: 23 - protein, 12 aa, feline coronavirus
TPQRRSRTSTSD

SEQ ID NO: 24 - protein, 12 aa, feline coronavirus
TRPRRSRRSTSE

SEQ ID NO: 25 - protein, 11 aa, feline coronavirus
PRVGQRSAVED

SEQ ID NO: 26 - protein, 11 aa, feline coronavirus
PHIGQRSAVED

SEQ ID NO: 27 - protein, 12 aa, feline coronavirus
TRPGRSRRSTSE

SEQ ID NO: 28 - protein, 12 aa, feline coronavirus
TSSKRSLRSTPA

SEQ ID NO: 29 - protein, 11 aa, feline coronavirus
PIVGKRSAVED

SEQ ID NO: 30 - protein, 11 aa, feline coronavirus
PRIGQRSAVED

SEQ ID NO: 31 - protein, 12 aa, feline coronavirus
TRPRRSRTSTSE

SEQ ID NO: 32 - protein, 11 aa, feline coronavirus
PRVGKRSAVED

SEQ ID NO: 33 - protein, 12 aa, feline coronavirus
TRPRRSRGSTSE

SEQ ID NO: 34 - protein, 12 aa, feline coronavirus
TQSKRSRRSTPA

SEQ ID NO: 35 - protein, 12 aa, feline coronavirus
TLSRRSRRLTSE

SEQ ID NO: 36 - protein, 11 aa, feline coronavirus
PRVGMRSTVED

SEQ ID NO: 37 - protein, 12 aa, feline coronavirus
TSSRSSRRSTSE

SEQ ID NO: 38 - protein, 11 aa, feline coronavirus
SRIGERSAVED

SEQ ID NO: 39 - protein, 12 aa, feline coronavirus
SQSRRSRSSTSE

SEQ ID NO: 40 - protein, 12 aa, feline coronavirus
THSRRSRGSTST

SEQ ID NO: 41 - protein, 12 aa, feline coronavirus
TQAKRSRRPTSH

SEQ ID NO: 42 - protein, 11 aa, feline coronavirus
PKIGVRSAVED

SEQ ID NO: 43 - protein, 12 aa, feline coronavirus
TQARRSRSSTPN

SEQ ID NO: 44 - protein, 11 aa, feline coronavirus
PKIGKRSAVED

SEQ ID NO: 45 - protein, 11 aa, feline coronavirus
PKIGARSAVED

SEQ ID NO: 46 - protein, 12 aa, feline coronavirus
TSARKSRRSVTE

SEQ ID NO: 47 - protein, 11 aa, feline coronavirus
LRIGKRSAVED

SEQ ID NO: 48 - protein, 11 aa, feline coronavirus
PRVGMRSAVED

SEQ ID NO: 49 - protein, 12 aa, feline coronavirus
TSARKSLRSVTE

SEQ ID NO: 50 - protein, 11 aa, feline coronavirus
PRVGKSSAVED

SEQ ID NO: 51 - protein, 11 aa, feline coronavirus
PRIGKRSAVED

SEQ ID NO: 52 - protein, 12 aa, feline coronavirus
TQPRQSRRSANP

SEQ ID NO: 53 - protein, 6 aa, feline coronavirus
RRSRSS

SEQ ID NO: 54 - protein, 12 aa, feline coronavirus
HTSRRSRGSAPN

SEQ ID NO: 55 - protein, 12 aa, feline coronavirus
HHSRRSRGSAPN

SEQ ID NO: 56 - protein, 12 aa, feline coronavirus
TSSRRSPRSTLD

SEQ ID NO: 57 - protein, 12 aa, feline coronavirus
PQPRRARMSVPE

SEQ ID NO: 58 - protein, 12 aa, feline coronavirus
THSRRSRMSTQN

SEQ ID NO: 59 - protein, 12 aa, feline coronavirus
THSRRSLRSTQN

SEQ ID NO: 60 - protein, 12 aa, feline coronavirus
TQSRRASTSTSN

SEQ ID NO: 61 - protein, 12 aa, feline coronavirus
HSRRRASTSTSN

SEQ ID NO: 62 - protein, 12 aa, feline coronavirus
TSSRRSLRSTVR